(19) United States
(12) Patent Application Publication
(10) Pub. No.: US 2007/0106164 A1
(43) Pub. Date: May 10, 2007

(54) Sphygmomanometer capable of setting user-defined standard blood pressure values

(75) Inventor: Paul Yang, Taipei (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD, CHUNG HO CITY (TW)

(21) Appl. No.: 11/581,416

(22) Filed: Oct. 17, 2006

(30) Foreign Application Priority Data
Nov. 7, 2005 (DE) 20 2005 017 370.3

(51) Int. Cl. A61B 5/02

(52) U.S. Cl. 600/485; 600/490

(57) ABSTRACT

A sphygmomanometer includes a pressure measuring unit connected to an analog-to-digital conversion unit, and the analog-to-digital conversion unit is connected to a microprocessor, and the microprocessor is connected separately to a setup unit, display unit, data storage unit, reminding unit and a power supply unit. The analog-to-digital conversion unit converts a blood pressure measured by the pressure measuring unit into data. Meanwhile, the microprocessor retrieves and compares the standard value of blood pressure pre-stored in the data storage unit with the measured value and displays the result on the display unit and the reminding unit. Users can change the predetermined setup of standard blood pressure values through the setup unit and users can understand the meaning of the complicated measured data through different colors or sounds of the backlight module of the reminding unit without memorizing the values, and determine whether the measured value is normal or critical.

### BACKGROUND OF THE INVENTION

1. Field of the Invention

The present invention relates to a sphygmomanometer capable of setting user-defined standard blood pressure values, and more particularly to a sphygmomanometer that allows users to use a setup unit to set the standard values of measured blood pressures based on the health reference standard values recently revised by the World Health Organization (WHO), the American Joint National Committee (JNC) on Prevention, Detection, Evaluation, and Treatment of High Blood Pressure or related medial organizations or the standard measured blood pressure values recommended by medical professionals according to the patient's medical history for reminding the user by different colors or sounds provided by a backlight module of a reminding unit, so that users need not remember all the complicated measured values to know about the message of a normal condition or a severe condition by different colors or sounds provided by the backlight module of the reminding unit.

2. Description of the Related Art

Since the concept of homecare becomes increasingly popular, quicker and cheaper, patients do not need medical professionals to operate measuring instruments such as blood sugar meters, electronic ear thermometers, electronic sphygmomanometers, and the like in order to know about their health conditions, and thus saving the time and costs for paying a visit to a medical clinic or a hospital and taking physiological measurements. Users can simply use their own medical measuring instruments to know about their health conditions anytime and anywhere to prevent any delay of the patient's condition.

However, general users are not medical professionals and usually are not familiar with the actual meanings of the measured values displayed by various different medical instruments. Although some medical instrument manufacturers build standard values and critical values of blood pressure into certain medical instruments according to the values specified by the World Health Organization (WHO), the American Joint National Committee (JNC) on Prevention, Detection, Evaluation and Treatment of High Blood Pressure or related medical organizations and indicate the values with light signals in different colors or warning sounds to remind users about a normal condition or a severe condition, yet the World Health Organization (WHO), the American Joint National Committee (JNC) on Prevention, Detection, Evaluation and Treatment of High Blood Pressure or related medical organizations may revise or amend the standard values and critical values of blood pressure based on the development of medical science and clinical experience. For instance, the Seventh Report of Joint National Committee (JNC7) amended the standard value of blood pressure in 2003 and the Sixth Report of Joint National Committee (JNC6) amended the systolic pressure/diastolic pressure<140/90 mmHg to the standard blood pressure values of systolic pressure/diastolic pressure<120/80 mmHg, and thus users have to get used to the recently amended standard and critical values. However, manufacturers build the instruments with a reminding device with a factory default setting or a predetermined value and do not provide users a way of altering the values according a new standard, and thus easily causing misjudgments or delays of a patient's conditions.

Since each user may have a measured reading different from the announced standard values due to the genetic factors of the family or the conditions of illness, such as the user is a hypertension patient, whose average measured blood pressure is higher than the blood pressure of a normal person. Without any seriously abnormal problem, the critical value should be adjusted to a high value for reminding the severe condition, so as to comply with the average measured condition. Since the predetermined critical reminding value set by the manufacturer is adjusted, unnecessary panic to users may result. For instance, a dialysis patient usually has an average measured blood pressure lower than the average blood pressure of a normal person, and such user should adjust the reminding value to a lower value in order to comply with the average measured blood pressure.

In the meantime, a general traditional sphygmomanometer provides light signals in different colors and allows users to determine whether or not their measured blood pressure values are standard blood pressure values. For instance, a hypertension patient having systolic pressure/diastolic pressure<120/80 mmHg is indicated by the display of a green light signal, or a pre-hypertension patient having systolic pressure/diastolic pressure =120˜139/80˜89 mmHg is indicated by the display of a yellow light signal, or a severe hypertension with systolic pressure/diastolic pressure>140/90 mmHg is indicated by a red light signal. For dialysis patients and hypotension patients, their measured blood pressure values are lower than the average blood pressures of normal people, such that if the measured blood pressure value is lower than a certain value, the reminding message for a severe condition cannot be provided. Since the reminding lamps usually come with a small volume and a weak brightness of the light source and those frequently using a sphygmomanometer to measure blood pressures are elderly people or patients, their visions are not as good, and thus they may have troubles to distinguish the colors of the light signals.

### SUMMARY OF THE INVENTION

In view of the shortcomings of the traditional sphygmomanometers, the inventor of the present invention aimed at the problem of the traditional sphygmomanometers to conduct extensive researches and experiments, and finally invented a sphygmomanometer capable of setting user-defined standard blood pressure values in accordance with the present invention.

The primary objective of the present invention is to provide a sphygmomanometer capable of setting user-defined standard blood pressure values, comprising a setup unit for setting new standard values and critical values according to the standard values and critical values revised by related health organizations or the instructions given by medical professionals, so as to reflect the actual conditions of each user.

Another objective of the present invention is to provide a sphygmomanometer capable of setting user-defined standard blood pressure values that comprises a pressure measuring unit coupled to an analog-to-digital conversion unit and the analog-to-digital conversion unit is coupled to a microprocessor, and the microprocessor is coupled separately to a setup unit, a display unit, a data storage unit and a power supply unit, such that when the sphygmomanometer is used and the pressure measuring unit measures a blood pressure, the blood pressure will be converted into data by an analog-to-digital conversion unit. In the meantime, a microprocessor is provided for users to set appropriate personal standard values of the blood pressure by a setup unit, so that the standard value or critical value can be taken from a data storage unit for comparison, and a reminding unit is provided for reminding users by a reminder corresponding to a predetermined setup, and users can know their physical conditions in a simple and easy manner.

A further objective of the present invention is to provide a sphygmomanometer capable of setting user-defined standard blood pressure values, comprising a microprocessor, so that users can set a critical reminding value and compare such value with the standard blood pressure value specified by related health organizations. If the newly set critical reminding value is higher than the standard blood pressure value, the user is determined as a hypertension patient, such that if the blood pressure value measured in the future is higher than the standard blood pressure value, the reminding unit will remind the user by a reminder corresponding to a predetermined setup. If the newly set critical reminding value is lower than the standard blood pressure value, the user is determined as a hypotension patient, such that if the measured blood pressure is lower than the standard blood pressure value, then the reminding unit will remind the user by a reminder corresponding to a predetermined setup.

Another further objective of the present invention is to provide a sphygmomanometer capable of setting user-defined standard blood pressure values, comprising a display unit which can be an LCD module, and the measured result and the patient's measured data can be displayed from the LCD module.

Another objective of the present invention is to provide a sphygmomanometer capable of setting user-defined standard blood pressure values, comprising a reminding unit which can be a backlight module for displaying different colors, and the measured results are displayed in different colors to allow users to know about the normal condition, the observing condition or the severe condition of the user's blood pressure.

Another objective of the present invention is to provide a sphygmomanometer capable of setting user-defined standard blood pressure values, comprising a reminding unit which can be a backlight module for displaying different colors.

### BRIEF DESCRIPTION OF THE DRAWINGS

To make it easier for our examiner to understand the objective, shape, assembly, structure, characteristics and performance of the present invention, the following embodiments accompanied with the related drawings are described in details.

FIG. 1 is a perspective view of the present invention;

FIG. 2 is a block diagram of the present invention; and

FIG. 3 is a flow chart of the present invention.

### DETAILED DESCRIPTION OF THE PREFERRED EMBODIMENTS

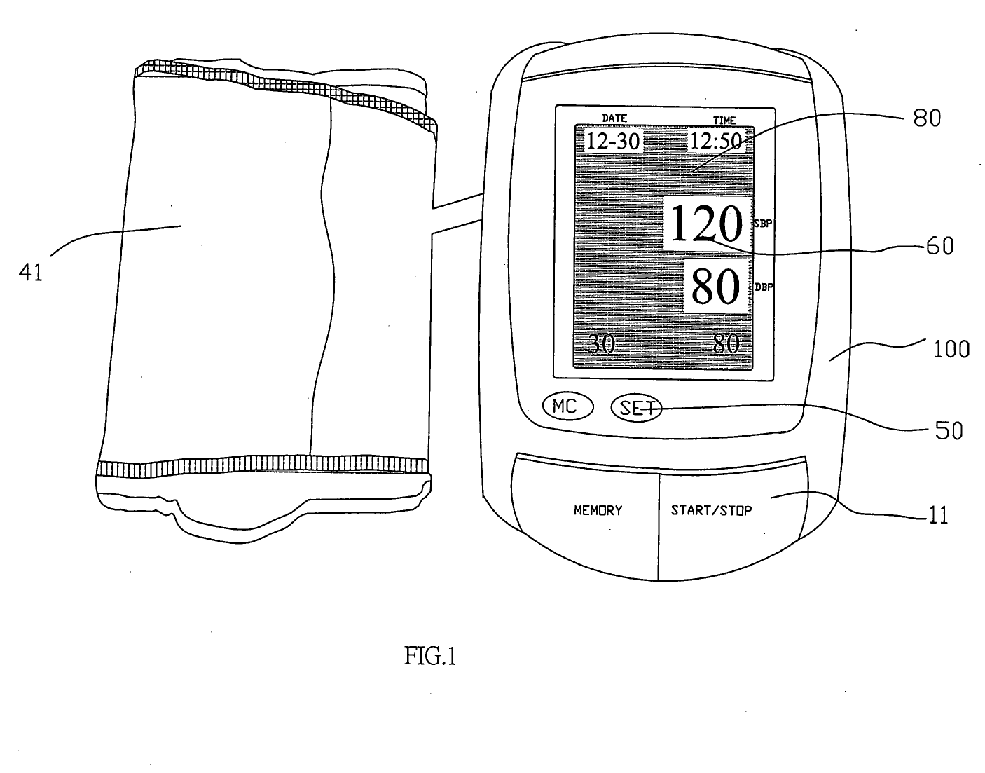

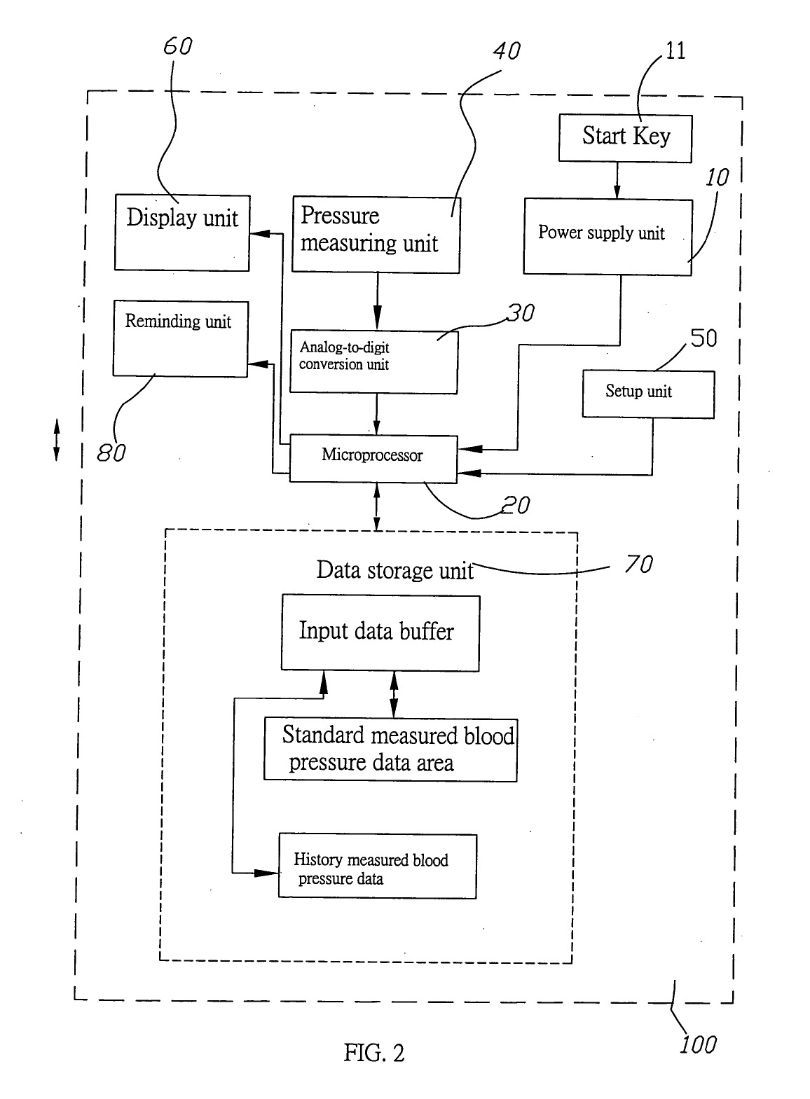

Referring to FIGS. 1 and 2 for a sphygmomanometer capable of setting user-defined standard blood pressure values in accordance with the present invention, the sphygmomanometer includes a casing 100 which can be of any shape, and the casing 100 has a power supply unit 10 coupled to a start key 11 disposed on the casing 100, so that when the sphygmomanometer is used, the start key 11 is turned on to start related measuring devices.

The casing 100 includes a microprocessor 20 coupled to a power supply unit 10, and the microprocessor 20 is coupled to an analog-to-digital conversion unit 30 and the analog-to-digital conversion unit 30 is coupled to a pressure measuring unit 40, and the pressure measuring unit 40 is coupled to an air cuff 41, and the air cuff 41 is worn on a human wrist or arm, such that when the air cuff 41 is inflated to compress the arteries of the arm or wrist and measure the pulse of the human arteries, and the analog-to-digital conversion unit 30 converts the blood pressure signal measured by the pressure measuring unit 40 into a digital signal.

The microprocessor 20 is coupled to a setup unit 50, a display unit 60, a data storage unit 70 and a reminding unit 80, wherein the display unit 60 is an LCD module for displaying results; the setup unit 50 is a press key for altering the setup for the standard values of different blood pressures; the data storage unit 70 is a memory, and the data storage unit 70 is provided for storing preset standard values of blood pressure and the history measure values of blood pressure; and the reminding unit 80 is a backlight module, such that the measure results can be displayed in different colors through a backlight module of the reminding unit 80. If a user sets the value according to the standard value and critical value of blood pressure modified by related health organizations, and the newly set reminding value is higher than the standard blood pressure value, then the user is determined as a hypertension patient, such that if the blood pressure value measured in the future is higher than the standard blood pressure value, then the microprocessor 20 will remind the user through the reminding unit 80 by a reminder corresponding to a predetermined setup. For instance, a standard blood pressure value (systolic pressure/diastolic pressure<120/80 mmHg) is indicated by a backlight module displayed in green, or a pre-hypertension (systolic pressure/diastolic pressure=120˜139/80˜89 mmHg) is indicated by a backlight module displayed in yellow, or a severe hypertension (systolic pressure/diastolic pressure>140/90 mmHg) is indicated by a backlight module displayed in red. If the newly set reminding value is lower than the standard blood pressure value, the user is determined as a hypotension patient, such that if the measured blood pressure value is lower than the standard blood pressure value, then the reminding unit 80 will remind the user by a reminder corresponding to a predetermined setup. For instance, the standard blood pressure value (systolic pressure/diastolic pressure<120/80 mmHg) is indicated by a backlight module with a green display, or the systolic pressure/diastolic pressure=110˜100/70˜75 mmHg is indicated by a backlight module displayed in yellow, or the severe hypotension (systolic pressure/diastolic pressure<100/70 mmHg) is indicated by a backlight module displayed in red.

Referring to FIGS. 1 to 3 for the application of the sphygmomanometer, the power supply is turned on such that the power supply unit 10 will supply power components connected to the sphygmomanometer. In the meantime, an air cuff 41 is worn at a human arm or wrist, and a start key 11 is pressed for starting and measuring the blood pressure, such that a pump (not shown in the figure) will inflate the air cuff 41 and the air cuff 41 will compress the arteries of the arm or wrist. After an appropriate pressure is inflated, the pump is turned off, and a pressure release value (not shown in the figure) is started to slowly and stably release the pressure of the air cuff 41 at the pressure measuring unit 40 for measuring the systolic pressure (SBP) and the diastolic pressure (DBP) of the blood in human arteries, and the data measured by the pressure measuring unit 40 is sent the microprocessor 20 through the analog-to-digital conversion unit 30. If the microprocessor 20 receives the data, the data will be displayed on the display unit 60 and the data pre-stored in the data storage unit 70 is retrieved and compared. If such data is not greater than or less than the predetermined value, then the data will be displayed on the display unit 60 and the reminding unit 80 adopts a green backlight module for the display. If such data is greater than or less than the predetermined value, then the microprocessor 20 will issue a warning message to remind the user through the reminding unit 80 by a predetermined warning color (yellow or red) of the backlight module, and the measured result will be stored in the data storage unit.

If the standard value and critical value of blood pressure are amended by the World Health Organization (WHO), the American Joint National Committee (JNC) on Prevention, Detection, Evaluation, and Treatment of High Blood Pressure or related medical organizations or instructed by doctors, users can set the values again through the setup unit 50. In the meantime, the newly set warning values is displayed by the reminding unit 80, and the values are stored into the data storage unit 70. If a measurement is made, the newly set standard value is compared with the measured data, and the result is displayed by the display unit 60 and the reminding unit 80 obtains any corresponding warning, and the measured blood pressure data is recorded and provided to doctors for reference.

In summation of the above description, the sphygmomanometer capable of setting user-defined standard blood pressure values in accordance with the present invention herein enhances the performance and overcomes the shortcoming of the prior art, and further complies with the patent application requirements.

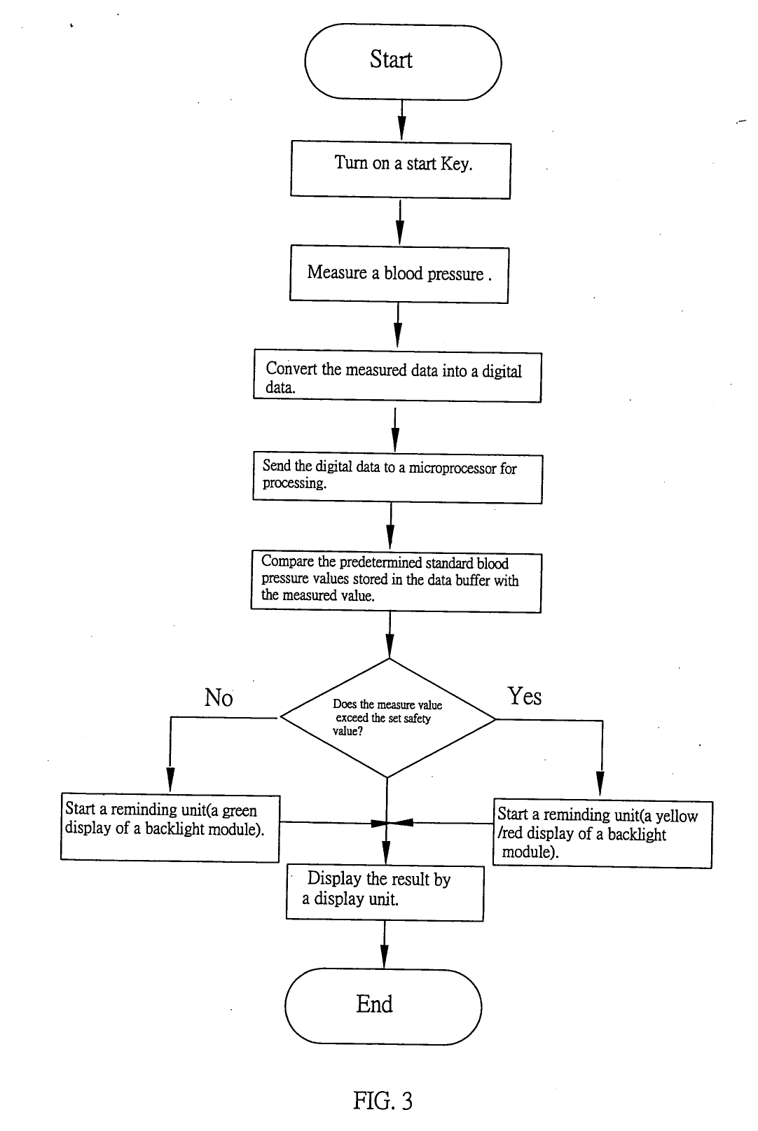

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.
10 Power supply unit
11 Start key
20 Microprocessor
30 Analog-to-digit conversion unit
40 Pressure measuring unit
50 Setup unit
60 Display unit
70 Data storage unit
80 Reminding unit
   Input data buffer
Standard measured blood pressure data area
History measured blood pressure data
FIG. 2
   Start
Turn on a start key.
Measure a blood pressure.
Convert the measured data into a digital data.
Send the digital data to a microprocessor for processing.
Compare the predetermined standard blood pressure values stored in the data buffer with the measured value.
Does the measure value exceed the set safety value? Yes/No
Start a reminding unit (a green display of a backlight module).
Start a reminding unit (a yellow/red display of a backlight module).
Display the result by a display unit.
End
FIG. 3

What is claimed is:

1. A sphygmomanometer capable of setting user-defined standard blood pressure values, comprising:
a pressure measuring unit, for measuring human arterial pressure;
an analog-to-digital conversion unit, coupled to said pressure measuring unit, for converting an analog signal of a measured blood pressure value into a digital signal;
a microprocessor, coupled to said analog-to-digital conversion unit, for determining and processing;
a display unit, coupled to said microprocessor, for displaying a result;
a setup unit, coupled to said microprocessor, for changing various different related setup of blood pressure standard values;
a data storage unit, coupled to said microprocessor, for storing different variables of a reference background and measured results;
a reminding unit, coupled to said microprocessor, for reminding users about the comparison of a measured blood pressure value and a predetermined standard value by different corresponding color displays of said backlight module; and
a power supply unit, coupled to said pressure measuring unit, said microprocessor, said display unit and said setup unit, for supplying a required power;
thereby, when a user's blood pressure is measured, said setup unit sets a new standard value according to actual conditions and based on standard values and critical values of blood pressure modified by the World Health Organization (WHO), the American Joint National Committee (JNC) on Prevention, Detection, Evaluation, and Treatment of High Blood Pressure or related medical organizations or instructed by doctors; and in the meantime, a user-defined standard value is compared with the standard value set by related medical organizations to determine whether said user is a hypertension patient or a hypotension patient, and said values are stored in said data storage unit, such that when a blood pressure is measured, said recently set standard value is compared with measured data, and the result is displayed by said display unit and reminded by said reminding unit.

2. The sphygmomanometer capable of setting user-defined standard blood pressure values of claim 1, wherein said setup unit is a press key.

3. The sphygmomanometer capable of setting user-defined standard blood pressure values of claim 1, wherein said display unit is a liquid crystal display module.

4. The sphygmomanometer capable of setting user-defined standard blood pressure values of claim 1, wherein said data storage unit is an external memory.

5. The sphygmomanometer capable of setting user-defined standard blood pressure values of claim 1, wherein said reminding unit corresponds to different color displays of a backlight module by different sounds.
